(19) United States
(12) Patent Application Publication
(10) Pub. No.: US 2024/0344942 A1
(43) Pub. Date: Oct. 17, 2024

(54) METHOD FOR PREPARING SAMPLE FROM SPECIMEN

(71) Applicants: JICHI MEDICAL UNIVERSITY, Tokyo (JP); EIKEN KAGAKU KABUSHIKI KAISHA, Tokyo (JP)

(72) Inventors: Kazutaka FUJITA, Shimotsuke-shi (JP); Koichi HAGIWARA, Shimotsuke-shi (JP)

(21) Appl. No.: 18/631,687

(22) Filed: Apr. 10, 2024

(30) Foreign Application Priority Data
Apr. 14, 2023 (JP) 2023-066507

(51) Int. Cl. G01N 1/30; C12N 15/10; C12Q 1/6886

(52) U.S. Cl. CPC G01N 1/30; C12N 15/1003; C12Q 1/6886

(57) ABSTRACT

A method for preparing a sample from a specimen includes treating a biopsy tissue taken from a lung cancer patient with negative pressure in an isotonic solution to release a cell from the biopsy tissue, collecting the isotonic solution containing the released cell, and mixing the collected isotonic solution and a cytologic specimen collected from the lung cancer patient together to obtain a cell suspension, wherein the cytologic specimen is one or more selected from the group consisting of a bronchial lavage fluid, a bronchoalveolar lavage fluid, a bronchial scraping lavage fluid and a puncture needle lavage fluid, and wherein the biopsy tissue and the cytologic specimen contain a tumor cell.

### CROSS-REFERENCE TO RELATED APPLICATIONS

This application is based upon and claims the benefit of priority from Japanese Patent Application No. 2023-066507, filed on Apr. 14, 2023, the entire contents of which are incorporated herein by reference.

### BACKGROUND

### Field

The present disclosure relates to a method for preparing a sample from a specimen.

### Description of the Related Art

Among lung cancers, it is said that 60% or more of adenocarcinomas have driver gene mutations, which directly affect canceration and, for Asians, 70% or more of adenocarcinomas have the mutations. Various molecular target drugs have been developed for driver gene mutations, and a lot of successful results have been achieved. Therefore, in order to determine a treatment method for an unresectable advanced or recurrent non-small-cell lung cancer, it is essential to investigate the presence or absence of driver gene mutations after the confirmation of pathological diagnosis. In addition, even for a resectable non-small-cell lung cancer, if the cancer is in a disease stage where the chance of recurrence is suspected, investigating the presence or absence of driver gene mutations in advance is one option.

In the test for driver gene mutations, a testing method in which a single gene mutation is detected has long been used. In recent years, various driver gene mutations have been discovered and numerous molecular target drugs therefor have been developed, and thus, multiple single-gene mutation tests have become necessary, and the lack of the amount of specimens has been acknowledged in a not insignificant number of cases. At the same time, a method for simultaneously detecting a multiple-gene mutation by next-generation sequencing (NGS) has been developed, and the test methods for driver gene mutations are transitioning to this method.

The present inventors developed MINtS (the Mutation Investigator using Next-era Sequencer), which is a multiple-gene mutation test system using a next-generation sequencer as the main component (PLOS ONE. 2017 Apr. 27; 12(4)). MINtS detects, from a cytologic specimen, an epidermal growth factor receptor (EGFR), a Kirsten rat sarcoma viral oncogene homolog (KRAS), a v-RAF murine sarcoma viral oncogene homolog B1 (BRAF) and an erb-b2 receptor tyrosine kinase 2 (ERBB2) gene within DNAs and detects an anaplastic lymphoma kinase (ALK), a ROS proto-oncogene 1, receptor tyrosine kinase (ROS1), and a ret proto-oncogene (RET) fusion genes within RNAs, and enables the determination of treatment regimens based on the gene mutation test in fully-advanced lung cancer patients. By narrowing down the target to a very small number of genes compared to gene panels that detect hundreds of genes, it has become possible to reduce the price through simultaneous testing of many patients and to improve the precision due to the increased amount of data per gene. As a result of a prior study using 4000 or more specimens, it is presumed that, compared to large gene panels, a price reduction to about 1/10 per sample and 10 times or more improvement in sensitivity can be expected.

However, the detection of multiple gene mutations by next-generation sequencing targets unstained specimen slides of cancer tissues fixed by immersion in a 10% neutral buffered formalin solution, cytologic specimens (such as washing fluids or fine needle aspirates), or plasma cell-free DNAs from blood specimens, and outdated samples or specimens are being used.

### SUMMARY

It is widely known that formalin used in the fixation process of cancer tissues causes the chemical and physical modification of nucleic acids or proteins and has an extremely significant influence on the quality of a sample. Therefore, in the guidelines, guidance and the like related to the treatment effect-prediction test (commonly known as companion diagnostics), a review of work procedures is underway. The test for multiple gene mutations using the next-generation sequencing is affected by a number of factors such as the pre-fixation process factors including the time from resection to fixation and the tissue size, the fixation process factors including the composition and concentration of the formalin fixative, pH, and the time and temperature of fixation, and the post-fixation process factors including the paraffin infiltration conditions and the storage temperature of the formalin-fixed paraffin-embedded (FFPE) blocks, and thus, issues with sample quality which have not often appeared in the single-gene mutation detection have been acknowledged in the multiple-gene mutation detection by the next-generation sequencing.

With cytologic specimens such as washing fluids and fine needle aspirates, if the number of tumor cells is small or the specimen has deteriorated, the collection of a sufficient amount of neoplastic nucleic acids cannot be expected. Similarly, with plasma cell-free DNAs from blood specimens, the less advanced the stage of the disease, the less likely it is to collect neoplastic nucleic acids. If the disease stage is somewhat advanced, such as with multiple distant metastases, the collection of neoplastic nucleic acids from blood can be expected, but even then, the collection rate is often confined to about 50% to 70%.

The present disclosure has been made in consideration of the above, and an objective of the present disclosure is to prepare a sample suitable for genetic analysis by the next-generation sequencing.

Typically, an alveolar tissue obtained from a lung biopsy is inflated by applying negative pressure in a syringe containing sterile saline before being fixed in formalin and embedded in paraffin, and then subjected to a pathological diagnosis. Here, the saline in the syringe is discarded after negative pressure is applied to the alveolar tissue. The present inventors took particular note of the fact that a small amount of a tumor cell is floating in this saline and found that the cells can be used for genetic analysis by the next-generation sequencing, and thus, completed the present disclosure.

Disclosed herein are the following aspects of the invention.
[1] A method for preparing a sample from a specimen, the method including:
treating a biopsy tissue taken from a lung cancer patient with negative pressure in an isotonic solution to release a cell from the biopsy tissue;
collecting the isotonic solution containing the released cell; and
mixing the collected isotonic solution and a cytologic specimen collected from the lung cancer patient together to obtain a cell suspension,
wherein the cytologic specimen is one or more selected from the group consisting of a bronchial lavage fluid, a bronchoalveolar lavage fluid, a bronchial scraping lavage fluid and a puncture needle lavage fluid, and
wherein the biopsy tissue and the cytologic specimen contain a tumor cell.
[2] The method according to [1], further including:
treating a cell in the cell suspension with a solution containing ammonium sulfate; and collecting the cell from the solution containing ammonium sulfate to obtain a sample.
[3] The method according to [1], further including:
treating a cell in the cell suspension with a solution containing ammonium sulfate to extract a nucleic acid from the cell; and
collecting the extracted nucleic acid to obtain a sample.
[4] A method for analyzing a gene, including:
performing a genetic analysis by next-generation sequencing using a sample prepared by the method according to [2] or [3].

According to the present disclosure, it is possible to prepare a sample suitable for genetic analysis by the next-generation sequencing. Specifically, in the present disclosure, since there is no need of treatments of formalin fixation and paraffin embedding for the preparation of a sample, it is possible to suppress the degradation of nucleic acids or cells in the sample. Therefore, a sample prepared by the method of the present disclosure can be said to be suitable for genetic analysis by the next-generation sequencing at least in that there is less degradation of nucleic acids or cells. The sample prepared by the method of the present disclosure can be said to be suitable for genetic analysis by the next-generation sequencing also in that the yield of nucleic acids is greater compared to that of cytologic specimens and the amplification of nucleic acids tends to be favorable. Furthermore, according to the method of the present disclosure, it is possible to conveniently prepare a sample within a short period of time. When combined with a multiple-gene mutation detection method by the next-generation sequencing, the present disclosure brings a great benefit to the determination of the suitability of a molecular target drug and can be said to be beneficial to the future medical science.

### BRIEF DESCRIPTION OF THE DRAWINGS

FIG. 1 is a graph showing the evaluation results of the quality of the samples on which genetic analysis has been performed in Test Example 1.

FIG. 2 is a graph showing the detection results of the EGFR gene mutations in Test Example 2.

FIG. 3A and FIG. 3B are graphs showing the relationship between the DNA yield and the quality of the samples in the samples with a DNA yield of 450 ng or less in Test Example 2.

FIG. 4A and FIG. 4B are graphs showing the second sample/first sample read ratios in the total read count of the normal sequence of the EGFR gene and in the total read count of the mutant sequence (FIG. 4A) and the second sample/first sample read ratio in the total read count of the EML4-ALK fusion gene sequence (FIG. 4B) in Test Example 3.

FIG. 5A and FIG. 5B are graphs showing the detection results of the EGFR gene mutations in cases where “excellent” (FIG. 5A) or “poor amplification” (FIG. 5B) first samples were obtained in Test Example 4.

FIG. 6A and FIG. 6B are graphs showing the detection results of the EGFR gene mutations in cases where “cell necrosis” (FIG. 6A) or “poor” (FIG. 6B) first samples were obtained in Test Example 4.

### DETAILED DESCRIPTION

A method for preparing a sample from a specimen according to one aspect of the present disclosure includes
(1) treating a biopsy tissue taken from a lung cancer patient with negative pressure in an isotonic solution to release a cell from the biopsy tissue,
(2) collecting the isotonic solution containing the released cell, and
(3) mixing the collected isotonic solution and a cytologic specimen collected from the lung cancer patient together to obtain a cell suspension (mixing step).

The obtained cell suspension, cells and nucleic acids that are obtained by further treating this cell suspension, and suspensions and solutions thereof are all within the scope of the “sample” prepared by the method according to the present aspect.

In the present specification, the term “sample” means a substance that is obtained by chemically or physically treating a specimen taken from a patient (a biopsy tissue, a cytologic specimen or the like). The sample prepared by the method according to the present aspect may be specifically a sample for genetic analysis, and may be specifically a cell, a nucleic acid, or a suspension or solution thereof. The sample prepared by the method according to the present aspect is suitable for genetic analysis by the next-generation sequencing at least in that it has less degradation, and is particularly suitable for a multiple-gene mutation analysis by the next-generation sequencing.

The biopsy tissue is not particularly limited as long as the biopsy tissue includes a tumor cell, and biopsy tissues that are normally used for a pathological diagnosis of lung cancer patients may be used. The specimens (tissues) that have been taken from patients and then chemically or physically processed, such as tissues fixed in formalin and/or embedded in paraffin, are not included in the scope of the biopsy tissue. The biopsy tissue may be a biopsy specimen or a surgical specimen. The biopsy tissue may be, for example, a lung, a lymph node or a metastatic tumor. When the biopsy tissue is a biopsy specimen, the biopsy method is not particularly limited and may be, for example, a bronchoscopic biopsy, a percutaneous biopsy or a thoracoscopic biopsy, and may be a forceps biopsy, a needle biopsy or a cryobiopsy.

The isotonic solution is not particularly limited, and known isotonic solutions that are normally used for the preservation of cells such as saline or phosphate buffered saline may be used.

A method for the treatment with negative pressure is not particularly limited as long as it is a method that induces a pressure change sufficient to cause a release of some cells from the biopsy tissue, and the same method as the known method for re-inflating an alveolar tissue after biopsy and before formalin fixation may be used. In this method, the biopsy tissue is treated with negative pressure in a syringe. Specifically, the biopsy tissue is first placed inside the outer barrel of the syringe filled with the isotonic solution, the plunger is then pushed to expel the air from the outer barrel, and subsequently, the plunger is pulled to apply negative pressure to the biopsy tissue in the outer barrel. By applying negative pressure to the biopsy tissue, some cells in the biopsy tissue are released into the isotonic solution. In the present specification, the isotonic solution containing the released cells will be referred to as “negative pressure collection liquid” in some cases.

A method for collecting the isotonic solution containing the released cells is not particularly limited. As one example, in a case where the treatment with negative pressure is performed in the syringe, the isotonic solution may be collected by transferring the isotonic solution to a separate container while leaving the biopsy tissue in the syringe.

The cytologic specimen is one or more selected from the group consisting of a bronchial lavage fluid, a bronchoalveolar lavage fluid, a bronchial scraping lavage fluid and a puncture needle lavage fluid, and it contains a tumor cell. The cytologic specimen may be, for example, a combination of a bronchial lavage fluid or a bronchoalveolar lavage fluid and a bronchial scraping lavage fluid.

In the mixing step (3), the cytologic specimen and the isotonic solution are usually mixed in equal amounts, but the proportion of the cytologic specimen may be decreased. For example, the cytologic specimen and the isotonic solution may be mixed at a volume ratio of 1:1 to 4.

The cell suspension obtained in the mixing step (3) contains a biopsy tissue-derived cell and a cytologic specimen-derived cell. The biopsy tissue-derived cell and the cytologic specimen-derived cell preferably contain a tumor cell. The cell suspension itself may be used for genetic analysis. Namely, a nucleic acid may be extracted, collected and, if necessary, purified from the cell suspension by a conventionally known method, and genetic analysis may be performed on the obtained nucleic acid. Alternatively, the cell in the cell suspension may further be treated with a solution containing ammonium sulfate, and the obtained cell or a nucleic acid thereof may be collected and used for genetic analysis.

Namely, in one embodiment, the method for preparing a sample from a specimen may further include
(4a) treating a cell in the cell suspension obtained in the mixing step (3) with a solution containing ammonium sulfate (treatment step), and
(5a) collecting the cell from the solution containing ammonium sulfate to obtain a sample. The solution containing ammonium sulfate quickly permeates into the cell to inactivate ribonucleases, deoxyribonucleases and proteases, and can suppress the degradation of nucleic acids, primarily RNA.

The concentration of ammonium sulfate in the solution containing ammonium sulfate may be, for example, 20 to 100 w/v % or 30 to 80 w/v %. As the solution containing ammonium sulfate, commercially available products that have been put on the market as an RNA stabilizing solution, such as RNAlater (registered trademark) stabilizing solution (manufactured by Thermo Fisher Scientific) may be used.

From the viewpoint of the permeation of the solution containing ammonium sulfate into the cell, the time for treatment with the solution containing ammonium sulfate is preferably 30 minutes or longer and more preferably 1 hour or longer. According to the present inventors' new finding, ammonium sulfate has an action of extracting nucleic acids. Therefore, from the viewpoint of preventing the extraction of nucleic acids from the cell, the time for treatment with the solution containing ammonium sulfate is preferably 8 hours or shorter and more preferably 3 hours or shorter.

The treatment with the solution containing ammonium sulfate may be performed while the cell is suspended in the cell suspension, or may be performed after the cell is separated from the cell suspension by centrifugation or the like.

A method for collecting the cell from the solution containing ammonium sulfate is not particularly limited, and a known method such as centrifugation may be used. The collected cell sample can be used for genetic analysis. Namely, a nucleic acid may be extracted, collected and, if necessary, purified from the cell by a conventionally known method, and genetic analysis may be performed on the obtained nucleic acid.

In another embodiment, the method for preparing a sample from a specimen may further include
(4b) treating a cell in the cell suspension obtained in the mixing step (3) with a solution containing ammonium sulfate to extract a nucleic acid from the cell (treatment step), and
(5b) collecting the extracted nucleic acid to obtain a sample.

The details of the treatment step (4b) are the same as the treatment step (4a) in the above-described embodiment. However, in the treatment step (4b) of the present embodiment, since the extraction of a nucleic acid from the cell using the solution containing ammonium sulfate is required, the time for treatment with the solution containing ammonium sulfate is preferably extended.

From the viewpoint of extracting a nucleic acid from the cell, the time for treatment with the solution containing ammonium sulfate is preferably 3 hours or longer and more preferably 8 hours or longer. On the other hand, from the viewpoint of the stability of the nucleic acid extracted from the cell, the time for treatment with the solution containing ammonium sulfate is preferably 8 weeks or shorter and more preferably 4 weeks or shorter.

A method for collecting the extracted nucleic acid is not particularly limited, and a known method such as centrifugation may be used. For example, the solution containing ammonium sulfate may be centrifugalized to precipitate the cell, and a supernatant containing the nucleic acid may be collected as a sample. Genetic analysis may be performed on the collected nucleic acid sample after the sample is purified by a known method as necessary.

A method for analyzing a gene according to one aspect of the present disclosure includes performing genetic analysis by the next-generation sequencing using a sample prepared by the above-described method. The genetic analysis by the next-generation sequencing may be multiple-gene mutation analysis. In the present specification, the multiple-gene mutation analysis refers to a parallel analysis of multiple gene mutations in a single run. A gene mutation to be analyzed may be a mutation of a driver gene and may be, for example, a mutation of the EGFR, KRAS, BRAF or ERBB2 gene or a fusion gene of ALK, ROS1 or RET.

### Examples

### Test Example 1

Using biopsy tissues and cytologic specimens taken from 500 cases diagnosed with lung cancer by pathological diagnosis, samples for genetic analysis were prepared for each case as follows. As the biopsy tissues, biopsy specimens obtained through bronchoscopic biopsy, endobronchial ultrasound-guided transbronchial needle aspiration, needle biopsy of a metastatic tumor, thoracoscopic biopsy, computed tomography (CT)-guided needle biopsy, lymph node biopsy or cryobiopsy, or surgically resected tumor sections were used. As the cytologic specimens, combinations of bronchial lavage fluid or bronchoalveolar lavage fluid and bronchial scraping lavage fluid were used. First, the collected biopsy tissue was placed in a syringe filled with saline, and negative pressure was applied to release cells from the biopsy tissue into the saline. This is the same technique as the technique used to re-inflate alveoli in bronchoscopic biopsy specimens taken from patients with a diffuse lung disease. Next, the saline containing the released cells (hereinafter, referred to as “negative pressure collection liquid”) was collected, and the cytologic specimen and the negative pressure collection liquid were mixed at a volume ratio of 1:1 to 4 to obtain a cell suspension. This cell suspension was centrifugalized, and the supernatant was discarded to obtain a cell sediment. An RNAlater (registered trademark) stabilizing solution (manufactured by Thermo Fisher Scientific), which is a solution containing ammonium sulfate as a main component, was added to this sediment to resuspend the sediment. After 1 hour to 4 weeks, this suspension was centrifugalized, and a cell sediment (first sample) to be used for genetic analysis was collected.

Each sample was classified into the following three groups based on the results of the pathological diagnosis of the biopsy tissue and the cytologic specimen used in the preparation of the sample:
(A) A tumor cell is found in a tissue diagnosis, and a tumor cell is found in a cell diagnosis
(B) A tumor cell is found in a tissue diagnosis, but a tumor cell is not found in a cell diagnosis
(C) A tumor cell is not found in a tissue diagnosis, but a tumor cell is found in a cell diagnosis

Since the tumor cells contained in the samples of the Group B were all derived from the negative pressure collection liquids, the samples of the Group B can be regarded as samples prepared using the negative pressure collection liquid alone. Similarly, since the tumor cells contained in the samples of the Group C were all derived from the cytologic specimens, the samples of the Group C can be regarded as samples prepared using the cytologic specimen alone.

From the samples, nucleic acids were extracted and quantified using Maxwell (registered trademark) RSC Instrument: AS1400 or AS1390 (manufactured by Promega K.K.), and the mutations of driver genes in the nucleic acid extracts were analyzed by the MINtS method. Specifically, targeting the DNAs in the nucleic acid extracts, various mutations of the EGFR, KRAS, BRAF and ERBB2 genes were detected, and targeting the RNAs in the nucleic acid extracts, various fusion genes of ALK, ROS1 and RET were detected. The quality of the samples of each group was evaluated based on the nucleic acid yield and the read count (amplification amount) as follows:

Excellent: The yield of the extracted nucleic acid is 100 ng or more for DNA and 50 ng or more for RNA, and all target regions for DNA analysis have 400 reads or more.

Poor amplification: The yield of the extracted nucleic acid is 100 ng or more for DNA and 50 ng or more for RNA, and there is at least one target region for DNA analysis that have 100 or more and less than 400 reads.

Cell necrosis: The yield of the extracted nucleic acid is 100 ng or more for DNA and 50 ng or more for RNA, and all target regions for DNA analysis have less than 100 reads (namely, cell necrosis is suspected).

Poor: The yield of the extracted nucleic acid is less than 100 ng for DNA, or less than 50 ng for RNA.

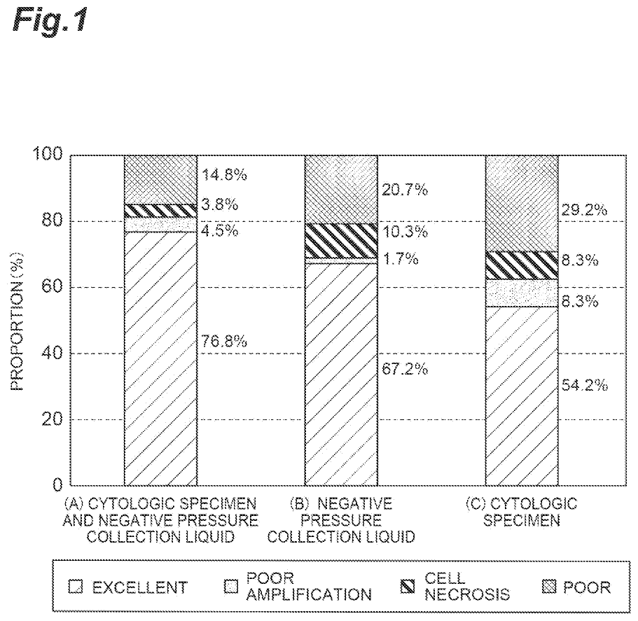

The results are shown in FIG. 1. The proportion of “excellent” samples was higher in the order of Group A, B and C. A higher proportion of “excellent” samples indicates that the target gene regions were well amplified. This suggests that when a mixture of negative pressure collection liquid and a cytologic specimen is used as a sample for genetic analysis, the nucleic acid yield becomes larger and the nucleic acid amplification becomes favorable compared to using either the cytologic specimen or the negative pressure collection liquid alone.

### Test Example 2

Preparation of samples (first samples) and multiple-gene mutation analysis by the MINtS method were performed for the cases of Test Example 1 in the same manner as in Test Example 1. The quality (excellent, poor amplification, cell necrosis or poor) of the samples were evaluated in the same manner as in Test Example 1 based on the nucleic acid yield and the read count (amplification amounts). However, the multiple-gene mutation analysis by the MINtS method were performed only on samples with the extracted nucleic acid yield of 100 ng or more for DNA and 50 ng or more for RNA (case count: 419).

For comparison, formalin-fixed paraffin-embedded (FFPE) unstained slides were prepared from the biopsy tissues of the 419 cases, and various mutations of the EGER gene were detected by conventional companion diagnostics targeting a single gene mutation. The conventional companion diagnosis was performed using the cobas (registered trademark) method, PNA-LNA (peptide nucleic acid-lock nucleic acid) PCR clamp method, PCR-INVADER (registered trademark) method or therascreen (registered trademark).

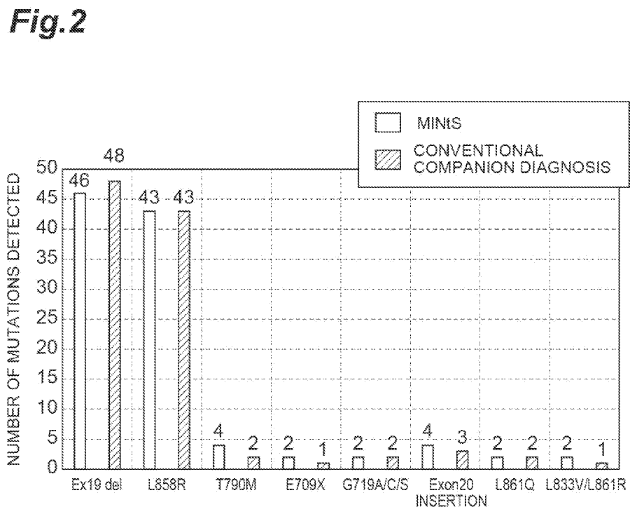

The detection results of the EGER gene mutations are shown in FIG. 2. The number of mutations detected using the MINtS method with the samples prepared in the same manner as in Test Example 1 was equivalent to that detected using the conventional companion diagnostics with the conventional unstained FFPE slides.

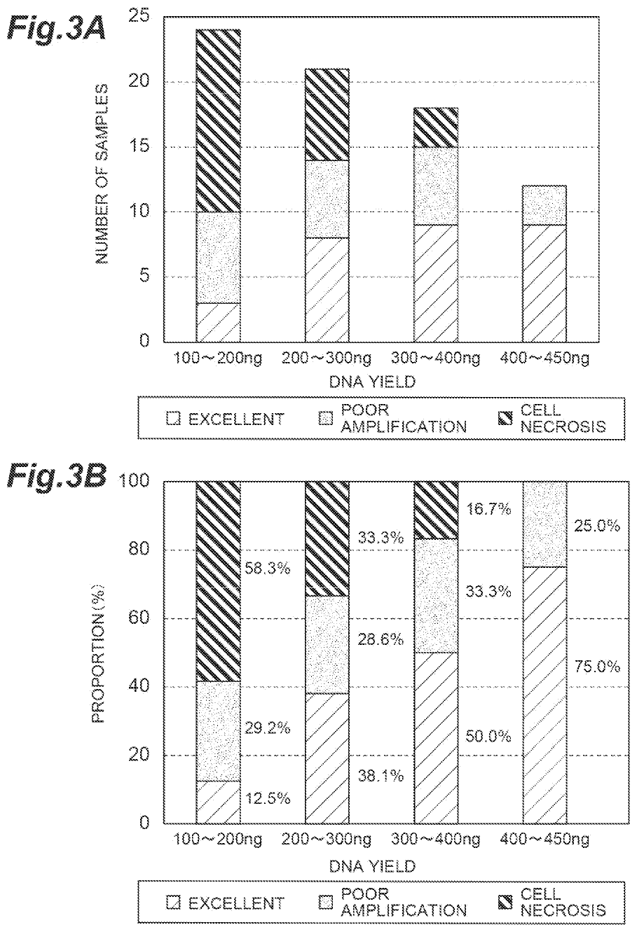

The DNA yield of the samples analyzed by the MINtS method was generally 10³ to 10⁴ ng, but there were also samples with a DNA yield of 450 ng or less. The number and proportion of the samples evaluated as excellent, poor amplification or cell necrosis among the samples with a DNA yield of 450 ng or less are shown in FIG. 3A and FIG. 3B, respectively. When the DNA yield was as small as 450 ng or less, the number and proportion of “excellent” samples decreased (that is, the amplification of the analysis target region decreased), with the decrease in the DNA yield (further investigated in Test Example 4).

### Test Example 3

Cultured tumor cells with mutations in driver genes were cultured and mixed with NCI-H23 strains with no mutations such that the proportion of the tumor cells with mutations became 10%, 50% or 100%. As the cultured tumor cells with mutations in driver genes, HCC827 strain (mutation: EGFR exon 19 deletion) or NCHI-H1975 strain (mutation: EGFR exon 21 L858R and EGFR exon 20 T790M) having EGFR gene mutations or NCI-H2228 strain (two variants: v3a and v3b) having an EMI.4-ALK fusion gene were used. The obtained cell groups were suspended in 30 w/v % of ammonium sulfate solutions and centrifugalized after 0 days (the day of the suspension), 10 days and 17 days, and the sediments (first samples) and supernatants (second samples) were collected. It was confirmed by the observation of hemocytometers with an optical microscope and measurement with a plate reader that the second samples contained almost no cells (that is, less than 1.0×10⁴ cells/mL).

From the first samples, nucleic acids were extracted and purified using Maxwell (registered trademark) RSC Instrument, and EGFR gene mutations and EML4-ALK fusion genes in the nucleic acid extracts were analyzed by the MINtS method. Since it had been suggested by a preliminary investigation that nucleic acids derived from tumor cells were already contained in the second samples, the nucleic acid extraction treatment was not performed on the second samples, and only the nucleic acid purification treatment using Maxwell (registered trademark) RSC Instrument was performed, and the EGFR gene mutations and EMI.4-ALK fusion gene in the purified nucleic acids were analyzed by the MINtS method.

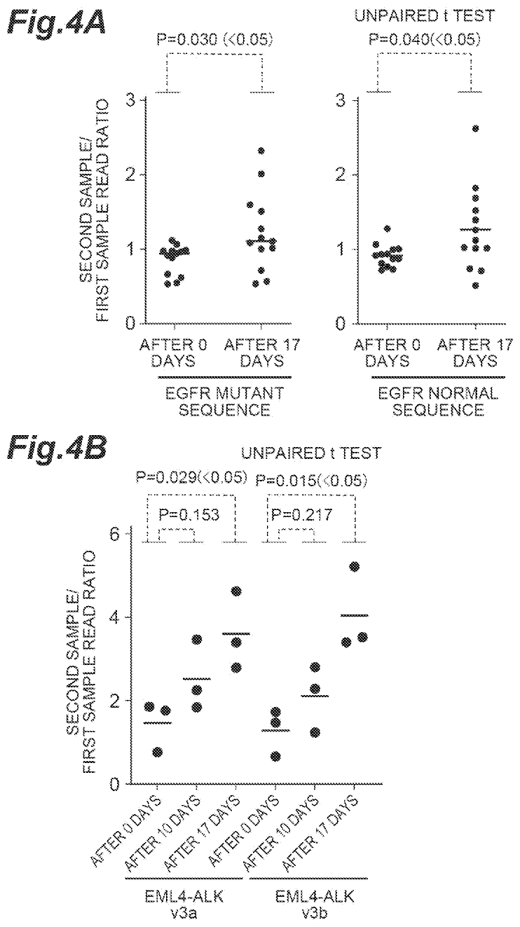

The ratio of the read count in the second sample to the read count in the first sample (second sample/first sample read ratio) was calculated for each of the total read count of the normal sequence and the total read count of the mutant sequences (sequences with the exon 19 deletion, exon 21 L858R mutation or exon 20 T790M mutation) of the analysis target region of the EGFR gene. The results are shown in FIG. 4A. In addition, the second sample/first sample read ratio was calculated for the total read count of the EML4-ALK fusion gene sequence. The results are shown in FIG. 4B.

For both sequences, the second sample/first sample read ratio increased as the time taken from the suspension of the cells in the ammonium sulfate solution to the centrifugation became longer. In this way, the nucleic acids were amplified even when using the second sample containing almost no cells, and furthermore, the amount of amplification increased as the preservation time of the cells in the ammonium sulfate solution increased. It was thus found that the ammonium sulfate solution has an action of extracting nucleic acids from cells. In addition, it was also found from these results that, as the preservation time of the cells in the ammonium sulfate solution becomes longer, the second sample (supernatant) becomes more suitable for genetic analysis than the first sample (sediment).

### Test Example 4

Mixtures of negative pressure collection liquid and a cytologic specimen were prepared from the cases of Test Example 1 in the same manner as in Test Example 1. The mixtures were centrifugalized, and supernatants were discarded to obtain cell sediments. An RNAlater (registered trademark) stabilizing solution was added to these sediments to resuspend the sediments. After 1 hour to 4 weeks, these suspensions were centrifugalized, and sediments (first samples) and supernatants (second samples) were collected.

Nucleic acids were extracted and purified from the first samples using Maxwell (registered trademark) RSC Instrument, and nucleic acids in the second samples were purified using Maxwell (registered trademark) RSC Instrument. EGFR gene mutations in the nucleic acids obtained from the first samples were analyzed by the MINtS method, and the quality (excellent, poor amplification, cell necrosis or poor) of the samples were evaluated in the same manner as in Test Example 1 based on the nucleic acid yield and the read count. The number of samples was 373 for “excellent” samples, 22 for “poor amplification” samples, 24 for “cell necrosis” samples and 81 for “poor” samples. Among these, in 119 cases of the cases that yielded the “excellent” samples, all cases that yielded the “poor amplification” or “cell necrosis” samples, and 73 cases of the cases that yielded the “poor” samples (total 238 cases), EGFR gene mutations in the nucleic acids obtained from the second samples were analyzed by the MINtS method.

For comparison, unstained FFPE slides were prepared from the biopsy tissues of the 238 cases, and various mutations of the EGFR gene were detected by conventional companion diagnostics targeting a single gene mutation. The conventional companion diagnosis was performed by the same method as the companion diagnosis in Test Example 1.

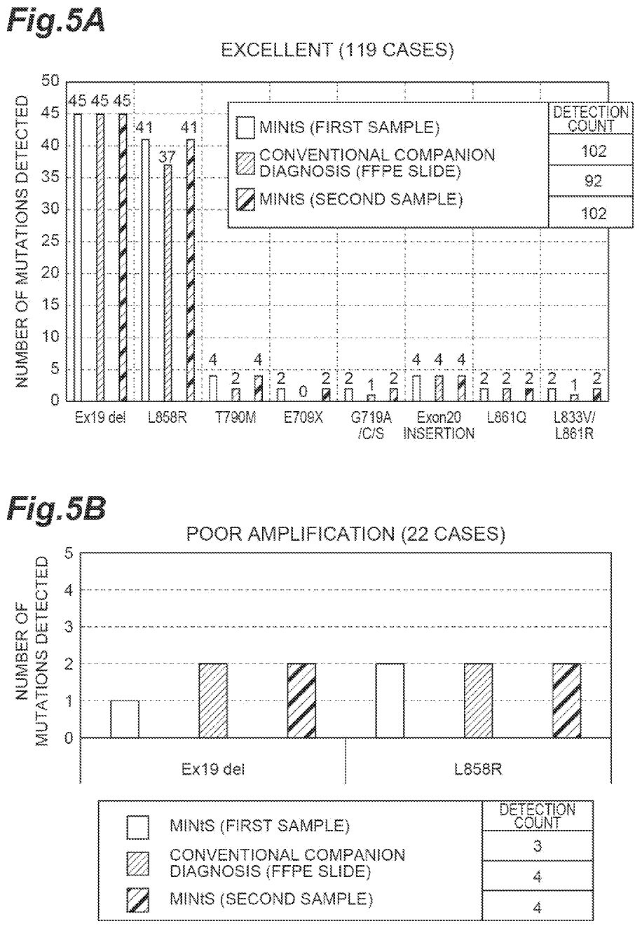

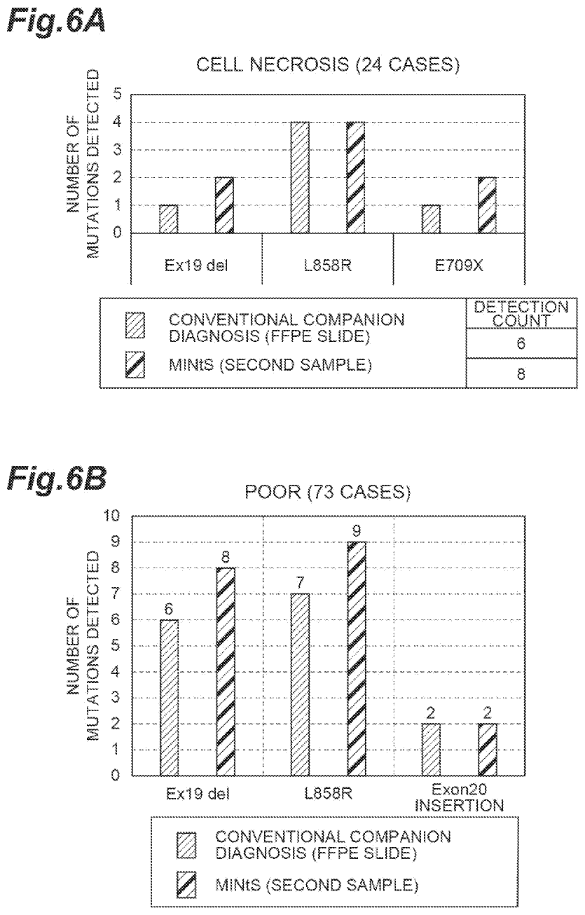

The detection results of mutations in the 238 cases are shown in FIG. 5A, FIG. 5B, FIG. 6A, and FIG. 6B. FIG. 5A and FIG. 5B show the results of the cases where the “excellent” or “poor amplification” first samples were obtained, and FIG. 6A and FIG. 6B show the results (only the results of the second samples and the unstained FFPE slides) of the cases where the “cell necrosis” or “poor” first samples were obtained. When the gene mutations were detected using the combination of the first sample or the second sample and the MINtS method, the number of mutations detected was equivalent to or more than that detected using the combination of the conventional unstained FFPE slide and the conventional companion diagnostics. In particular, it was confirmed that, when the gene mutations were detected using the combination of the second sample and the MINtS method, the number of mutations detected was equivalent to or more than that detected using the combination of the conventional unstained FFPE slide and the conventional companion diagnostics, even in the cases where the “cell necrosis” or “poor” first samples were obtained. Note that it has been confirmed that gene mutations in the second samples are not derived from blood components that may have contaminated the specimens.

It is to be understood that not all aspects, advantages and features described herein may necessarily be achieved by, or included in, any one particular example. Indeed, having described and illustrated various examples herein, it should be apparent that other examples may be modified in arrangement and detail.

What is claimed is:

1. A method for preparing a sample from a specimen, the method comprising:
treating a biopsy tissue taken from a lung cancer patient with negative pressure in an isotonic solution to release a cell from the biopsy tissue;
collecting the isotonic solution containing the released cell; and
mixing the collected isotonic solution and a cytologic specimen collected from the lung cancer patient together to obtain a cell suspension,
wherein the cytologic specimen is one or more selected from the group consisting of a bronchial lavage fluid, a bronchoalveolar lavage fluid, a bronchial scraping lavage fluid and a puncture needle lavage fluid, and
wherein the biopsy tissue and the cytologic specimen contain a tumor cell.

2. The method according to claim 1, further comprising:
treating a cell in the cell suspension with a solution containing ammonium sulfate; and
collecting the cell from the solution containing ammonium sulfate to obtain a sample.

3. The method according to claim 1, further comprising:
treating a cell in the cell suspension with a solution containing ammonium sulfate to extract a nucleic acid from the cell; and
collecting the extracted nucleic acid to obtain a sample.

4. A method for analyzing a gene, comprising:
performing a genetic analysis by next-generation sequencing using a sample prepared by the method according to claim 2.

5. A method for analyzing a gene, comprising:
performing a genetic analysis by next-generation sequencing using a sample prepared by the method according to claim 3.
